(19) United States
(12) Patent Application Publication
(10) Pub. No.: US 2005/0107959 A1
(43) Pub. Date: May 19, 2005

(54) Screening method for evaluation of bilayer-drug interaction in liposomal compositions

(76) Inventor: Yuanpeng Zhang, Sunnyvale, CA (US)

Correspondence Address: PERKINS COIE LLP, P.O. BOX 2168, MENLO PARK, CA, 94026, US

(21) Appl. No.: 10/956,815

(22) Filed: Oct. 1, 2004

Related U.S. Application Data
(60) Provisional application No. 60/508,344, filed on Oct. 3, 2003.

(51) Int. Cl. G06F 19/00; G01N 33/48; G01N 33/50; G01K 17/00

(52) U.S. Cl. 702/19; 374/30

(57) ABSTRACT

A method for generating a correlation between at least one thermal property of a liposomal carrier in the presence of a therapeutic agent and a pharmacokinetic property for the therapeutic agent in the liposomal carrier and using the correlation for predicting the pharmacokinetic property of the liposomal carrier in the presence of any therapeutic agent in a liposomal carrier.

This application claims the benefit of priority to U.S. Provisional Application No. 60/508,344, filed Oct. 3, 2003, which is incorporated herein by reference in its entirety.

### FIELD OF THE INVENTION

The invention relates to a screening technique to evaluate drug-lipid interactions using thermal measurements, such as with differential scanning calorimetry (DSC). This technique correlates thermal measurements to the biophysical data of various drugs loaded into STEALTH® liposomes with their respective pharmacokinetic data. A model was constructed that predicts the in vivo pharmacokinetic behavior of drugs loaded into STEALTH®, or long-circulating, liposomes to screen the potential of a drug in a lipidic delivery system, and provides a valuable tool to predict in vivo behavior of a given drug when administered from a liposomal platform.

### BACKGROUND OF THE INVENTION

Liposomes are closed lipid vesicles used for a variety of purposes, and in particular, for carrying therapeutic agents to a target region or cell by systemic administration of liposomes. Liposomes have proven particularly valuable to buffer drug toxicity and to alter pharmacokinetic parameters of therapeutic compounds. Conventional liposomes are, however, limited in effectiveness because of their rapid uptake by macrophage cells of the immune system, predominantly in the liver and spleen.

With regard to the short in vivo half-life of conventional liposomes, a number of companies have overcome this obstacle by designing liposomes that are non-reactive (sterically stabilized) or polymorphic (cationic or fusogenic). For example, the Stealth® liposome (Alza Corporation, Mountain View, Calif.) is sterically stabilized with a lipid-polymer moiety, typically a phospholipid-polyethylene glycol (PEG) moiety, is included in the liposomal bilayer to prevent the liposomes from sticking to each other and to blood cells or vascular walls. These liposomes appear to be invisible to the immune system and have shown encouraging results in cancer therapy (Haumann, Inform, 6:793-802, 1995). It has been shown that there is a positive correlation between the amount of liposomal drug accumulation in solid tumors and the blood circulation half-life of the liposomes. However, a challenge with these liposomes is that different drugs exhibit very different drug release profiles upon intravenous administration in vivo. Different drugs may exhibit different pharmacokinetic behaviors even when encapsulated inside the same type of liposomes by the same encapsulation method. Properties of both the lipid and the drug contribute to the drug retention and blood circulation making prediction of the drug retention difficult. However, little is known at present about how drugs interact with the lipid membranes, and furthermore, how the nature of the interaction affects drug leakage.

Therefore, achieving prolonged blood circulation for the liposome formulation is a primary focus in formulation feasibility studies, as the circulation half-life may directly relate to the efficacy of the product. Formulation feasibility studies include preparation of liposomes with an entrapped therapeutic agent and evaluation of pharmacokinetic (PK) data for the liposomes. Pharmacokinetic studies are designed to identify and describe one or more of absorption, distribution, metabolism and excretion of drugs. As the pharmacokinetic behavior of the free drug is very different from the same drug entrapped in a liposome, assessing the PK information is not straightforward. This evaluation is a lengthy process and usually takes 6 to 12 months to complete. One can not anticipate the outcome of the PK until the study is completed.

A model for identifying suitable carrier systems and predicting the performance of these systems was described by Barenholtz and Cohen (J Liposome Res., 5(4):905-932 (1995)). This system, however, has little use due to the multiple tasks for measuring parameters and does not provide a clear and direct prediction of the pharmacokinetic performance of the liposomal formulations, even with the knowledge of the values of these parameters.

Further, Hrynyk et al. proposed a mathematical model describing dose- and time-dependent liposome distribution and elimination to introduce a limited set of parameters, which may be helpful with assessing the in vivo fate of a liposomally encapsulated drug (Hrynyk et al., Cell Mol Biol Lett, 7(2):285, 2002).

It would be desirable, therefore, to predict the pharmacokinetics for a liposomal drug formulation. The present invention presents an empirical, predictive model based on an analytical technique such as differential scanning calorimetry. This predictive model is useful for drug screening in order to select drugs with a high potential for long circulation in liposome formulations as well as to identify drug candidates that are potentially problematic. Another use of the model is in designing appropriate lipid formulations for maximum blood circulation time.

### BRIEF DESCRIPTION OF THE DRAWINGS

FIGS. 1A-1D are graphs of thermograms of DSPC liposomes containing doxorubicin (FIG. 1A), CKD602 (FIG. 1B), vincristine (FIG. 1C), and paclitaxel (FIG. 1D) as compared to a DSPC control at a pH of 3.6;

FIGS. 2A-2D are graphs of thermograms of DSPC liposomes containing doxorubicin (FIG. 2A), CKD602 (FIG. 2B), vincristine (FIG. 2C), and paclitaxel (FIG. 2D) as compared to a DSPC control at a pH of 7.0.

FIG. 3 is a graph of a DSC thermograph for DSPC;

FIGS. 4A-4B are scatterplot matrix of correlations of ΔH_{VH} and CU, respectively, vs. blood circulation half-life in rats (T_{1/2}) for liposome entrapped drugs at pH 3.6;

FIGS. 5A-5B are bivariate scatterplot matrices of correlations of ΔH_{vH} vs. circulation half-life (T_{1/2}) for liposome entrapped drugs at pH 7.0;

FIGS. 6A-6B are multivariate scatterplot matrices of correlations for liposome entrapped drugs at pH 3.6 and 7.0, respectively.

### DETAILED DESCRIPTION OF THE INVENTION

I. Definitions

The terms below have the following meanings unless indicated otherwise.

“Liposomes” are vesicles composed of one or more concentric lipid bilayers which contain an entrapped aqueous volume. The bilayers are composed of two lipid monolayers having a hydrophobic “tail” region and a hydrophilic “head” region, where the hydrophobic regions orient toward the center of the bilayer and the hydrophilic regions orient toward the inner or outer aqueous phase.

“Vesicle-forming lipids” refers to amphipathic lipids which have hydrophobic and polar head group moieties, and which can form spontaneously into bilayer vesicles in water, as exemplified by phospholipids, or are stably incorporated into lipid bilayers, with the hydrophobic moiety in contact with the interior, hydrophobic region of the bilayer membrane, and the polar head group is moiety oriented toward the exterior, polar surface of the membrane. The vesicle-forming lipids of this type typically include one or two hydrophobic acyl hydrocarbon chains or a steroid group, and may contain a chemically reactive group, such as an amine, acid, ester, aldehyde or alcohol, at the polar head group. Included in this class are the phospholipids, such as phosphatidyl choline (PC), phosphatidyl ethanolamine (PE), phosphatidic acid (PA), phosphatidyl inositol (PI), and sphingomyelin (SM), where the two hydrocarbon chains are typically between about 14-22 carbon atoms in length, and have varying degrees of unsaturation. Also included within the scope of the term “vesicle-forming lipids” are glycolipids, such as cerebrosides and gangliosides.

“Hydrophilic polymer” as used herein refers to a polymer having moieties soluble in water, which lend to the polymer some degree of water solubility at room temperature. Exemplary hydrophilic polymers include polyvinylpyrrolidone, polyvinylmethylether, polymethyloxazoline, polyethyloxazoline, polyhydroxypropyloxazoline, polyhydroxypropyl-methacrylamide, polymethacrylamide, polydimethyl-acrylamide, polyhydroxypropylmethacrylate, polyhydroxyethylacrylate, hydroxymethylcellulose, hydroxyethylcellulose, polyethyleneglycol, polyaspartamide, copolymers of the above-recited polymers, and polyethyleneoxide-polypropylene oxide copolymers. Properties and reactions with many of these polymers are described in U.S. Pat. Nos. 5,395,619 and 5,631,018.

Abbreviations: DSC: Differential Scanning Calorimetry; PK: pharmacokinetic: T_{1/2}: blood circulation half-life, FTIR: Fourier Transform Infrared;: Cp: heat capacity:; Tm: phase transition temperature; ΔH_{vH}: van't Hoff's enthalpy;; CU: cooperativity or cooperative unit; PC: phosphatidylcholine; PG: phosphatidylglycerol; PS: phosphatidylserine; PA: phosphatidic acid; POPC: palmitoyloleoyl phosphatidylcholine; HSPC: fully hydrogenated soy PC; PHEPC: partially hydrogenated egg PC-IV40; EPC: egg phosphatidylcholine; DOPC: dioleoyl phosphatidylcholine; SOPC: stearyoyl oleoyl phosphatidylcholine; OPPC: oleolyl palmitoyl phosphatidylcholine; OSPC: oleoyl stearoyl phosphatidylcholine; DOPG: dioleoyl phosphatidylglycerol; DSPC: distearoyl phosphatidylcholine; PEG: polyethylene glycol.

II. Screening Method

A. Measurement of Thermal Properties

Many analytical methods and devices for measuring or determining thermal properties are known and used routinely in the art. Representative methods are discussed further below; however, it will be appreciated that any analytical technique that provides thermal data for a composition may be used herein.

1. Differential Scanning Calorimetry

Differential scanning calorimetry (DSC) is a method known in the art used to measure the amount of energy (as heat) absorbed or released by a sample as it is heated, cooled, or held at a constant temperature. As used herein, the term “DSC measurements” further includes calculations using a measured feature of the sample. An exemplary method of measuring DSC utilizes a differential scanning calorimeter. Any calorimeter is suitable as long as the temperature range of the calorimeter is appropriate for the sample measurements. An exemplary calorimeter is the VP-DSC differential scanning calorimeter available from MicroCal (Northampton, Mass., USA). Typical applications using the differential scanning calorimeter include determination of melting point temperature and/or the heat of melting, measurement of the glass transition temperature, curing and crystallization studies, and identification of phase transformations.

In the embodiment using a differential scanning calorimeter for measuring the thermodynamic properties of a lipid suspension, the heat flow into a sample is usually contained in a sample cell and measured differentially, i.e. by comparing the heat flow of the sample to the heat flow into an reference cell containing an equal volume of water or the aqueous component of the sample. The heat flow may be considered as the amount of heat (q) supplied per unit of time (t), or q/t. Typically, both cells sit inside a metal jacket with a known (calibrated) heat resistance (K). The temperature of the calorimeter is raised linearly with time (scanned), where the heating rate (β=dT/dt) of the cells are kept constant and consistent with each other. Any heating rate may be used, however the heating rate of the sample and reference cells must remain the same, or similar. The temperature may be controlled manually or automatically. In a preferred embodiment, the temperature control is automatic or computerized. Heat flows into the two cells by conduction from a heat source such as a radiator. The heat flow into the sample cell is larger due to the additional heat capacity (Cp) of the sample during the course of the phase transition. Heat capacity refers to the heat flow divided by the heating rate or Cp=q/ΔT, where q is heat, and ΔT is temperature increase. The difference in heat flow (dq/dt) induces a temperature difference (dT) between the sample and the reference cells. This temperature difference is measured using any appropriate sensor, such as a thermocouple, and a signal is generated representative of the difference.

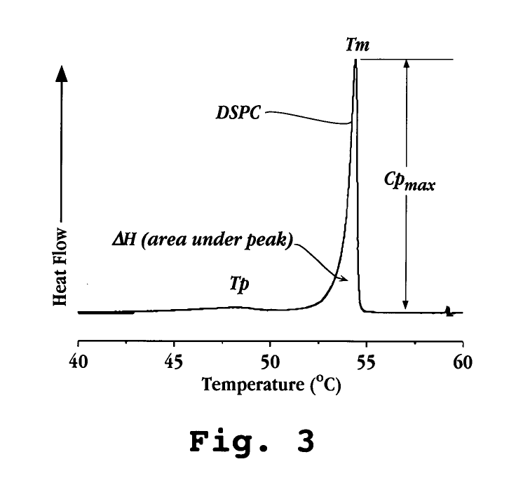

FIG. 3 depicts the thermogram of heat flow vs. temperature (° C.) for DSPC lipid vesicles without an entrapped drug showing the gel-to-liquid-crystal transition, also termed as the main phase transition. In this figure, in a heating scan, an endothermic event results in a positive (upward) deviation from the baseline. The major peak (Tm=54.4° C.) is associated with the main-phase transition (i.e. gel-to-liquid-crystal phase transition). The smaller peak (identified with Tp) is the pretransition. The melting temperature is seen on the heat flow plot as a peak as heat is absorbed by the sample until the phase transition is completed. As will be appreciated by those of skill in the art, the main phase transition extends over a temperature range, although this peak is typically very sharp for most vesicle-forming lipids, such as DSPC, the Tm can be reported as the onset of the transition, as the midpoint of the transition, the peak temperature, or any suitable point as long as the parameters are defined. The Tm is typically reported as either the maximum peak height of the transition or a point where a certain percentage of the phase transition has occurred, for example 40%, 50%, or 60% of the phase transition has occurred. It will be appreciated that the exact percent of phase transition is not important as long as it is defined. Where a ratio is used, it is desirable to use the same ratio for each sample to aid in comparison between the samples. In the studies reported herein, the Tm is reported as the maximum peak of the transition range.

With further reference to FIG. 3, above the Tm, the molecules of the sample become melted because lipid hydrocarbon chains are changing from a gel-like state to a fluid state.

The maximum heat capacity of the liposome (Cpₘₐₓ) relates to the heat capacity function at the peak temperature, Tm.

The latent heat of melting, or the calorimetric phase transition enthalpy, (ΔH_{cal}) can be determined by first determining the area (A) under the peak according to the following formula:
A=(heat in calories)(temperature in Kelvin)/(time in seconds)(mass in moles).
The latent heat of melting, i.e. the calorimetric enthalpy, may then be determined by the following:
ΔH_{cal}=(A/(q/t))m, where m is the mass of the sample (moles).

In other words, ΔH_{cal}, is defined as the area under the peak after baseline subtraction, scan rate normalization, and concentration normalization.

Measurements obtained by DSC may additionally be used to determine or calculate useful thermodynamic parameters for the sample, including the van't Hoff's enthalpy (ΔH_{vH}) and the cooperativity unit (CU). The van't Hoff's enthalpy is calculated from the following equation
ΔH_{vH}=(4R·Tₘ²·Cpₘₐₓ)/ΔH_{cal},

where Cpmax (kcal mol⁻¹ K⁻¹) is the heat capacity function at the phase transition peak after baseline-subtraction and concentration normalization, Tm (K) is the peak temperature, ΔH_{cal} is the calorimetric enthalpy defined as the integral of the heat capacity function after baseline subtraction, and R is the gas constant (1.987 cal mol⁻¹ K⁻¹) (Biocalorimetery: Applications of Calorimetry in the Biological Sciences, Ladbury and Chowdhry, Eds., John Wiley & Sons).

The cooperativity or cooperative unit (CU) is calculated with the following equation:
CU=ΔH_{vH}/ΔH_{ca}.

As will be illustrated below, both CU and ΔH_{vH} are useful for comparing the effect of entrapping drugs in a liposome on the thermodynamic properties of the lipid bilayer. As noted above, lipid bilayers are self-assembling macrostructures composed of a multitude of similar molecules (lipids). The phase transition upon heating or cooling of the lipid bilayer is a cooperative event among the lipid molecules. The CU can be considered a measure of the freedom of communication among the lipid molecules of the lipid bilayer.

It will be appreciated that other data can be determined from the DSC measurements including, but not limited to, the width of the phase transition at half-height (ΔTm_{1/2}), the full phase transition width (ΔTm), the transition temperature and enthalpy of the pretransition (Tp and ΔHₚ), etc.

In a preferred embodiment, high sensitivity DSC instruments are used because they can provide more sensitive and accurate phase transition profiles of the lipid. This may be important when the interactions of the drug and the lipid are weak and low sensitivity DSC instruments may not be adequate to resolve the fine changes in the phase transition profile.

2. Fourier Transform Infrared Spectroscopy

Fourier Transform Infrared Spectroscopy (FTIR) is an analytical technique typically used to identify organic inorganic materials. This technique measures the absorption of various infrared light wavelengths by the material of interest. This technique is used to identify thermal information for the material of interest, such as the main phase transition temperature and the phase transition width.

3. Electron Spectroscopy for Chemical Analysis

Electron Spectroscopy for Chemical Analysis (ESCA), also known as x-ray photoelectron spectroscopy or XPS, is a surface analysis technique used for obtaining chemical information about the surfaces of solid materials. The method utilizes an x-ray beam to excite a sample resulting in the emission of photoelectrons. An energy analysis of these photoelectrons provides thermal data for the sample (http://www.innovatechlabs.com).

It will be appreciated that any number of other analytical techniques or devices are suitable for measuring or determining the thermal property, including, but not limited to a simultaneous thermal analyzer (STA), a thermal mechanical analyzer (TMA), a dilatometer, thermogravimetry (TG or TGA), electron paramagnetic resonance (EPR), and a dynamic mechanical analyzer (DMA).

B. Correlation of Thermal Measurements

In one embodiment, the present method is useful for generating a correlation between at least one thermal property of a liposomal carrier in the presence of a therapeutic agent and a pharmacokinetic (PK) property. In a preferred embodiment, the method is useful for generating a correlation between at least one thermal property of a liposomal carrier in the presence of a therapeutic agent and the in vivo half-life. In another embodiment, the method is useful for generating a correlation between the in vivo half-life of a liposomal carrier in the presence of a therapeutic agent and the van't Hoff's enthalpy, the cooperative unit and/or the main phase transition temperature peak width. It will be appreciated that one of skill in the art is well acquainted with determination of pharmacokinetic properties through pharmacokinetic studies. Pharmacokinetic studies are briefly described below.

Pharmacokinetic studies are designed to identify and evaluate one or more of the basic pharmacological concepts: absorption (for extravascular administration), distribution, metabolism, and excretion. It will be appreciated that absorption properties of a drug by intravascular methods of administration, including intravenous administration, are not determined as the drug is administered directly to the blood and is, therefore, not absorbed to the blood stream. Further, the relationship between dose, plasma concentration, and therapeutic or toxic effects can be studied. Pharmacokinetic studies are used to evaluate the efficacy and toxicity of a therapeutic agent as well as to determine dosage, administration route, and scheduling for treatment. Pharmacokinetic studies of the rate of absorption, distribution, metabolism, and excretion generally can be determined from plasma/blood concentration over time data following administration.

Without being limited as to theory, it is believed that therapeutic agents that show increased interaction with the lipid affect the in vivo half-life of the liposome composition. The mechanism of drug release from STEALTH®) liposomes may be understood using Fick's law of diffusion:
J=−D*D C/Dx, where J is the drug efflux and D is the diffusion coefficient.

In order to prolong blood circulation (i.e., to reduce the drug efflux, J), one option is to decrease the value of diffusion coefficient, D, which can be achieved by using lipids that form solid bilayers. For a given lipid composition and liposome internal conditions, the drug diffusion coefficient is determined by the intrinsic nature of drug-lipid interactions. The other option to prolong blood circulation is to minimize the free drug concentration inside the liposomes, which can be achieved by forming drug precipitates using strong precipitation reagents.

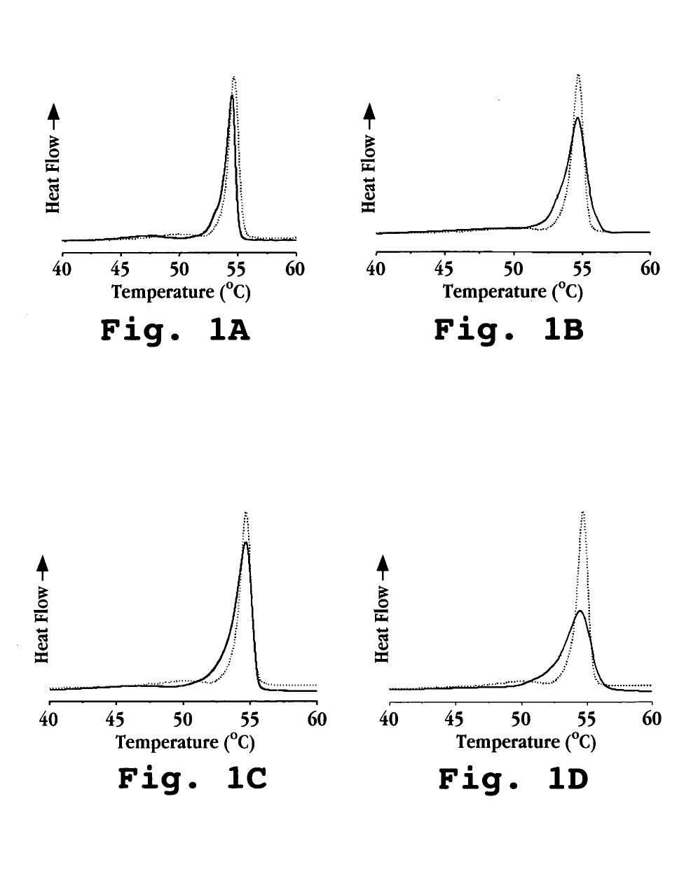

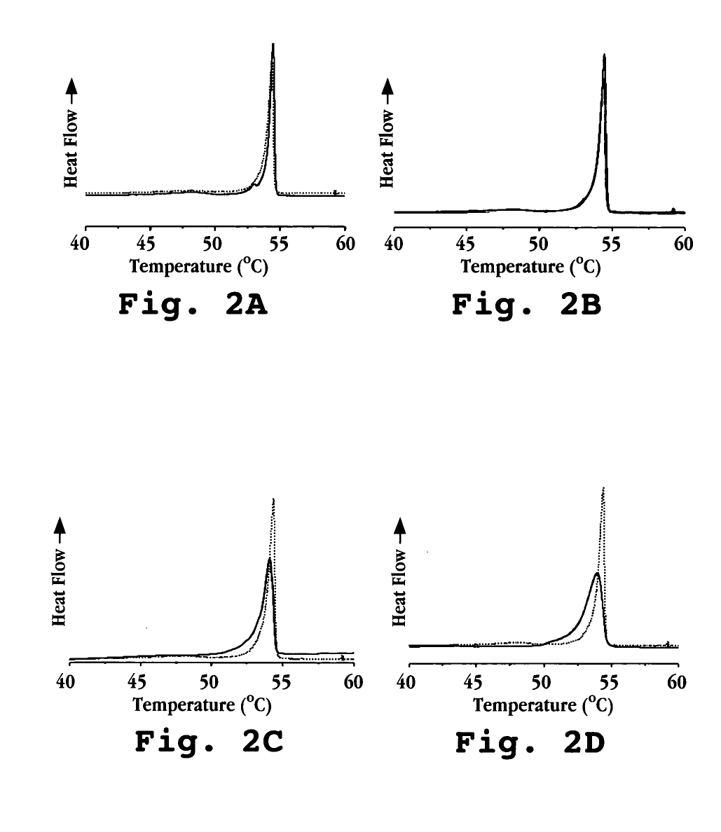

As detailed in Example 2, DSPC liposomes containing various drugs (doxorubicin, CKD602, vincristine, ciprofloxacin, or paclitaxel) were measured by VP-DSC. As seen in FIGS. 1A-1D, the effect on the phase transition of entrapping each of doxorubicin, CKD602, vincristine, ciprofloxacin, or paclitaxel in DSPC liposomes (solid line) was compared with a thermogram for empty DSPC liposomes as a control (dotted line). It should be noted that the liposomes prepared in accord with the present invention were formulated using pure lipids to reduce interference from the additional elements. DSPC was used in this study, because all STEALTH® liposomes are either prepared with DSPC or HSPC as the main bilayer forming lipid (except for paclitaxel). HSPC (fully hydrogenated soy PC) is very similar to DSPC with respect to its physical and chemical properties. Similar conclusions may be drawn if HSPC is used based on the example of DSPC. Cholesterol is also excluded in this study, because it is known that cholesterol significantly broadens the phase transition peak of phospholipids so that the effect of the presence of the drug will be totally lost. Use of these pure lipid formulations is, however, predictive of typical formulations including sterols such as cholesterol and of formulations including lipids derivatized with a hydrophilic polymer.

The in vivo blood circulation half-life (T_{1/2}) in rats upon intravenous injection for each of the drugs entrapped in STEALTH® liposomes is known and presented in Table 1 below along with the lipid compositions. The thermogram data for the drug-DSPC aqueous mixtures is presented in Tables 2a and 2b.

TABLE 1
Blood circulation half-life for STEALTH ® liposomes
with various drugs loaded and placebo liposomes
with ¹¹¹In as the radiolabel.

| | Lipid composition | T_{1/2} |
|---|---|---|
| Formulation | (mol/mol) | (hr) |
| STEALTH ® placebo | HSPC/CHOL/mPEG₁₉₀₀-DSPE | 24.6 |
| liposomes | (56.:38.9:5.3) | |
| doxorubicin liposomes | HSPC/CHOL/mPEG₁₉₀₀-DSPE | 26.5 ± 4.6 |
| | (56.4:38.9:5.3) | |
| CKD602 liposomes | DSPC/mPEG₁₉₀₀-DSPE (95:5) | 10.9 |
| vincristine liposomes | HSPC/CHOL/mPEG₁₉₀₀-DSPE | 10.3 |
| | (56.4:38.9:5.3) | |
| ciprofloxacin | HSPC/CHOL/mPEG₁₉₀₀-DSPE | 6.1 |
| liposomes | (50:45:53) | |
| paclitaxel liposomes | PHEPC/mPEG₁₉₀₀-DSPE (7.6:92.4) | 0.2 |

TABLE 2a
Blood circulation half-life of STEALTH ® liposome formulations and
thermodynamic parameters for DSPC with various drugs and placebo
liposomes with radiolabel ¹¹¹In at pH 3.6.

| | Tm | Tm | | | | | |
|---|---|---|---|---|---|---|---|
| drug | (° C.) | (K) | ΔHcal | Cpmax | ΔTm½ | ΔHvH | CU |
| DSPC | 54.7 | 327.8 | 9.44 | 8.5 | 0.92 | 769.2 | 81.5 |
| doxorubicin | 54.5 | 327.7 | 9.28 | 7.4 | 0.94 | 680.5 | 73.3 |
| CKD602 | 54.6 | 327.8 | 11.81 | 7.5 | 1.27 | 542.3 | 45.9 |
| vincristine | 54.6 | 327.8 | 12.77 | 7.1 | 1.42 | 474.9 | 37.2 |
| ciprofloxacin | 54.9 | 328 | 9.98 | 4.5 | 1.4 | 385.6 | 38.6 |
| paclitaxel | 54.5 | 327.6 | 10.6 | 3.5 | 2.27 | 281.7 | 26.6 |

TABLE 2b
Blood circulation half-life of STEALTH ® liposome formulations
and thermodynamic parameters for DSPC with various drugs and
placebo liposomes with radiolabel ¹¹¹In at pH 7.0.

| drug | Tm (C.) | ΔT½ | ΔHcal | Cpmax | ΔHv | Coop | Tm (K) |
|---|---|---|---|---|---|---|---|
| DSPC | 54.37 | 0.47 | 9.96 | 14.4 | 1231 | 124 | 327.52 |
| doxorubicin | 54.45 | 0.4 | 9.66 | 16.3 | 1437 | 149 | 327.6 |
| CKD602 | 54.43 | 0.42 | 11.2 | 17.2 | 1308 | 117 | 327.58 |
| vincristine | 53.93 | 1.38 | 11.65 | 6.81 | 496 | 43 | 327.08 |
| paclitaxel | 54.07 | 0.77 | 9.94 | 8.67 | 741 | 75 | 327.22 |

As seen in FIG. 1A, a comparison of the thermograms shows a similar phase transition curve for the DSPC/doxorubicin mixture (solid line) and the control liposomes (dotted line) indicating doxorubicin maintains a weak interaction with the bilayer. This data is consistent with the in vivo half-life for doxorubicin loaded STEALTH® formulations of about 26.5±4.6 hours (an average obtained from at least four separate studies), see Table 1. Similarly, as seen in FIG. 1D, the phase transition curve for the DSPC/paclitaxel mixture (solid line) shows significant deviation from the control DSPC (dotted line), indicating significant interaction of paclitaxel with the lipid bilayer. This deviation is reflected by a lower in vivo half-life of 0.2 hours. Thus, deviation of the sample curve from a control indicates a stronger interaction of the drug with the lipid bilayer. As seen in FIGS. 1B and 1C, the thermogram data for DSPC mixtures with CKD602, or vincristine (solid line) shows varying degrees of deviation from the DSPC control (dotted line) than the doxorubicin loaded liposomes, yet less deviation than the paclitaxel loaded liposomes. This data indicates CKD602 and vincristine each exhibit some interaction with the lipid bilayer. This middle deviation is reflected in an in vivo half-life between that known for doxorubicin loaded liposomes and paclitaxel loaded liposomes. It is expected that, in most cases, greater deviation from the control indicates greater interaction of the drug with the bilayer.

Hereafter, correlation of the DSC data with the in vivo half-life is discussed. However, it will be appreciated that correlation of the DSC data with another pharmacokinetic parameter such as AUC (area under the curve), clearance or the apparent volume of distribution is within the scope of the present method and within the skill of one in the art.

In one embodiment, the method includes generating a correlation between at least one thermal property of a liposomal carrier in the presence of a therapeutic agent and the in vivo blood circulation half-life of the liposomal carrier in the presence of a therapeutic agent. In this embodiment, the method includes measuring at least one thermal property of similar liposomal carriers in the presence of at least two therapeutic agents, separately. At least one reference correlating a range of in vivo blood circulation with the at least one thermal property is generated. In a preferred embodiment, the thermal property is measured by differential scanning calorimetry. It will be appreciated that a correlation generated for one liposomal carrier may be used to predict pharmacokinetic properties of a different liposomal carrier where the liposomal carriers are similar in structure and properties.

As described in Example 3, DSPC liposome formulations were formed containing paclitaxel, vincristine, CKD602, ciprofloxacin, or doxorubicin. DSC measurements were used to determine the main phase transition temperature (Tm), enthalpy (ΔHcal), heat capacity (Cp), and transition peak width at half-height (ΔTm1/2). The van't Hoff's enthalpy (ΔH_{vH}) and the cooperativity unit (CU) were calculated from the DSC measurements. The DSC measurements were made at two buffering conditions (pH 3.6 and pH 7.0) using the same drug-to-lipid mole ratio of 1:5 for each liposome composition. The DSC measurements were made at a temperature range of between 30-65° C. at a scan rate of 20° C./hour with the results shown in Tables 2a and 2b.

For the DSC data from Tables 2a and 2b, bivariate correlations were made for known T_{1/2} and the Tm, ΔH_{cal}, Cpₘₐₓ, ΔTm_{1/2}, ΔH_{vH}, and the CU with the results shown in Table 3 and 4, respectively. It will be appreciated that multivariate correlations may be made for any of the thermal data obtained with any pharmacokinetic property.

TABLE 3
Bivariate correlation at pH 3.6 analyzed using JMP 5.0.1a software
(SAS).

| | Tm (K) | ΔHcal | Cpmax | ΔTm½ | ΔHvH | CU |
|---|---|---|---|---|---|---|
| T½ | −0.1106 | −0.4920 | 0.8247 | −0.8863 | 0.9641 | 0.9690 |

TABLE 4
Bivariate correlation at pH 7.0 analyzed using JMP 5.0.1a software
(SAS).

| | Tm (K) | ΔT½ | ΔH | Cpmax | ΔH_{vH} | CU |
|---|---|---|---|---|---|---|
| T½ | 0.6665 | −0.4822 | −0.3839 | 0.6184 | 0.6943 | 0.7377 |

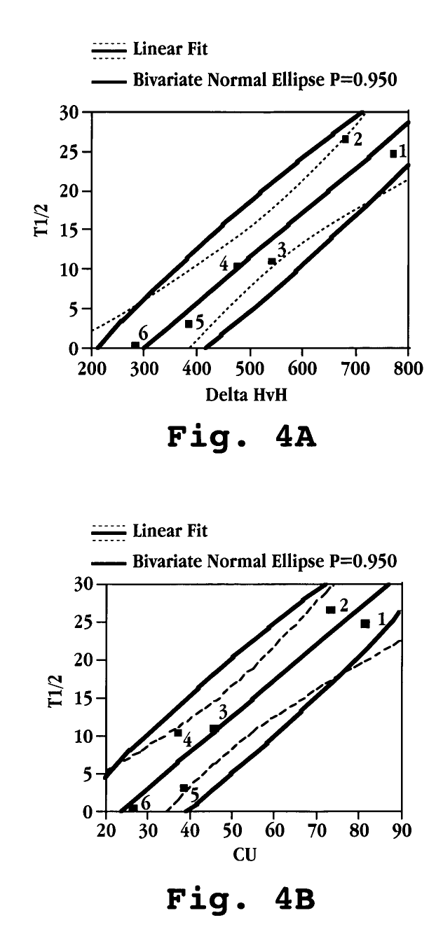

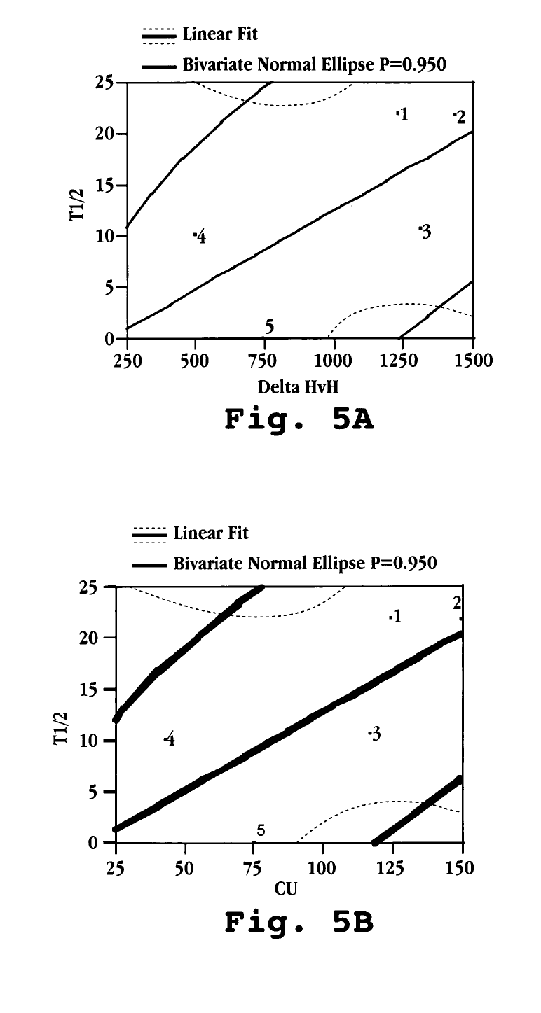

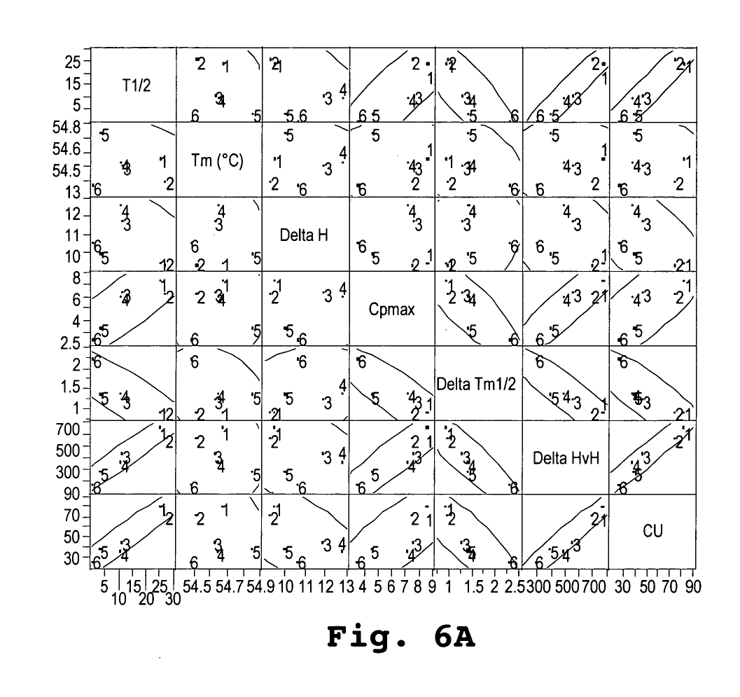

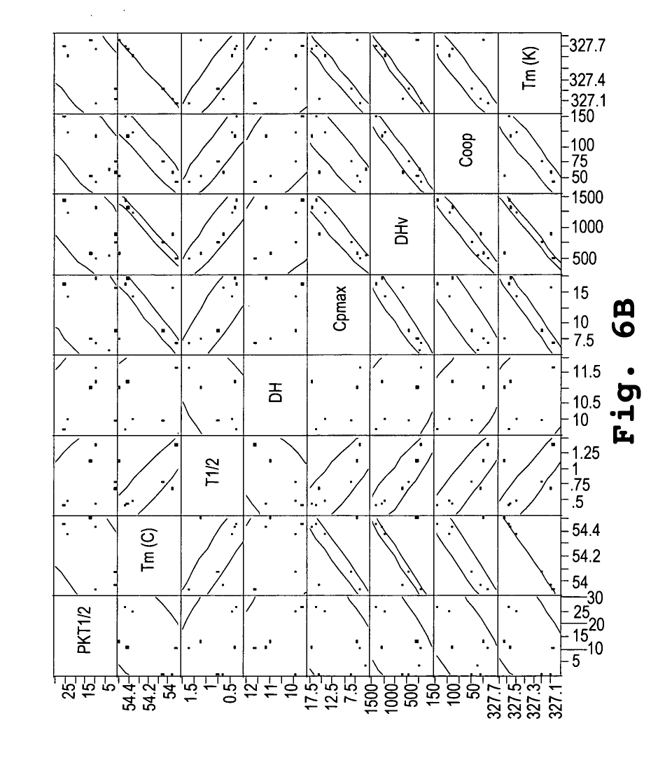

As seen from the above tables, ΔH_{vH}, CU, ΔTm_{1/2}, and Cpmax each showed significant correlation with the known in vivo half-life at pH 3.6. Without being limited as to theory, this may indicate the limiting step of drug leakage from liposomes is the partition of the drug molecules into the bilayer membrane from the liposomal internal aqueous core. As seen in FIGS. 4A-5B, bivariate scatterplots were prepared for the T_{1/2} vs. ΔH_{vH} and CU, respectively at pH 3.6 or 7.0. The results indicate that T_{1/2} has the best correlation with ΔH_{vH} at the low pH. This correlation may be used to predict the in vivo half-life of an unknown therapeutic agent if loaded into STEALTH® liposomes based on the ΔH_{vH} data generated for DSPC liposomes including an entrapped agent, where the in vivo half-life is known. It will be appreciated that one or more thermal properties may be correlated with the PK data for the purposes of this invention. As seen above there is also an excellent correlation between CU and T_{1/2} for the liposomes prepared in Example 3. It will further be appreciated that other methods for generating the correlation between the thermal property and the PK data are within the skill of one in the art. As seen in FIGS. 6A and 6B, multivariate scatterplots were prepared for the T_{1/2} vs. the DSC data for each of the liposomes prepared in Example 3.

As seen in FIGS. 4A-5B, or more clearly in the results in Example 3, a plot of PK in vivo half-life (in hours) versus either the ΔH_{vH} or the CU yields linear correlations.
T_{1/2}=−16.43+0.056 ΔH_{vH}
T_{1/2}=−8.054+0.429 CU

It will be appreciated that calculation of the slope of any correlation generated is well within the skill of one the art based on the y intercept using the equation y=mx+b, where x and y are coordinates of a point on the line and b is the y intercept. In one embodiment, the invention contemplates generation of a range based on the slope of the line. In one embodiment, this range deviates (+and/or −) about 10% from the actual slope of the line. In other embodiments, this range may deviate (+and/or −) about 15%, 20%, 25%, or more from the slope of the line.

As can be observed from the graphs, the higher the value of ΔH_{vH}, or CU, the greater the half-life observed. This curve, linear or otherwise, can be used to predict the in vivo half-life for potential liposomal carrier in the presence of the therapeutic agent. It has been well established that the lipid bilayer transition occurs substantially in unison unlike protein transition where the transition occurs in monomeric form giving rise to a broad transition peak. In the case of bilayer transition, it is believed that lipid molecules transition from gel-to-liquid crystalline form collectively and any deviation in the transition indicates the presence of a strong interaction of bilayers with ‘foreign material’ in the system.

As seen in FIGS. 5A and 5B, the data for pH 7.0 showed less significant correlation as compared to the data for pH 3.6, however insights regarding the interaction between the lipid bilayer and the drug may still be obtained from the data as well as a linear correlation for prediction of pharmacokinetic properties. Without being limited as to theory, it may be that drug dissociation from the outer surface of the liposome plays a lesser or weaker role in drug leakage or retention.

In another embodiment, the invention contemplates a method for predicting the in vivo blood circulation half-life of a liposomal carrier in the presence of a therapeutic agent. In this embodiment, a liposomal carrier is selected and at least one thermal property of the liposomal carrier in the presence of a therapeutic agent is determined by differential scanning calorimetry. A correlation is generated for the liposomal carrier. Thereafter, DSC measurements for a subsequent liposomal carrier in the presence of a therapeutic agent can be compared to the generated correlation to predict the in vivo half-life based on the correlation. It will be appreciated that the correlation may be generated as described above, or by any appropriate means.

### III. EXAMPLES

The following examples illustrate but are in no way intended to limit the invention.

Materials and Methods

DSPC was obtained from Avanti Polar Lipids, (Birmingham, Ala.).

### Example 1

### Liposome Preparation

The liposomes may be prepared by a variety of techniques, such as those detailed in Szoka, F., Jr., et al., (Ann. Rev. Biophys. Bioeng. 9:467 (1980)). Typically, the liposomes are multilamellar vesicles (MLVs), which can be formed by simple lipid-film hydration techniques. In this procedure, a mixture of liposome-forming lipids, including a vesicle-forming lipid derivatized with a hydrophilic polymer where desired, are dissolved in a suitable organic solvent which is evaporated in a vessel to form a dried thin film. The film is then covered by an aqueous medium to form MLVs, typically with sizes between about 0.1 to 10 microns. Exemplary methods of preparing derivatized lipids and of forming polymer-coated liposomes have been described in co-owned U.S. Pat. Nos. 5,013,556, 5,631,018 and 5,395,619, all of which are incorporated herein by reference.

The therapeutic agent can be incorporated into liposomes by standard methods, including (i) passive entrapment of a lipophilic compound by hydrating a lipid film containing the agent, (ii) loading an ionizable drug against an inside/outside liposome ion gradient, termed remote loading as described in U.S. Pat. Nos. 5,192,549 and 6,355, 268, both of which are incorporated herein by reference, and (iii) loading a drug against an inside/outside pH gradient. If drug loading is not effective to substantially deplete the external medium of free drug, the liposome suspension may be treated, following drug loading, to remove non-encapsulated drug.

### Example 2

### Preparation of DSPC Liposomes

Liposomes comprised of saturated phospholipid DSPC were prepared by thin-film hydration method as described in Example 1. Briefly, 6.3mM of lipid was weighed into a flask and dissolved in chloroform:methanol (9:1 v/v) mixture and the solvent mixture was evaporated at about 70° C. under vacuum using a rotavapor to form a uniform thin film of lipid. The lipid film was kept overnight at a high vacuum to ensure complete removal of solvent traces. The lipid film was hydrated at 60° C. using 2OmM of a phosphate buffer to obtain control liposomes.

For the preparation of doxorubicin, CKD602, vincristine, and ciprofloxacin (water-soluble drugs) loaded liposomes, the drug was dissolved in the hydrating buffer such that the resulting liposomes had a 1:5 lipid:drug ratio (mol/mol). For the preparation of paclitaxel (water-insoluble) loaded liposomes, the lipid and drug were co-dissolved in the solvent mixture such that the resulting liposomes had a 1:5 lipid:drug ratio (mol/mol). The resulting liposomes had a molar ratio of drug to lipid of 1 to 5. Free drug was not removed from the suspension.

### Example 3

### Differential Scanning Calorimetry Measurements and Statistical Analysis

Liposomes comprised of only DSPC were prepared as described in Example 2 with entrapped CKD602, doxorubicin, vincristine, ciprofloxocin, or paclitaxel.

DSC measurements were obtained with a VP-DSC available from MicroCal (Northampton, Mass.) at a heating rate of 20° C./hour. The data was analyzed using origin software and statistical software JMP5.0.1. The measurements were made of the drug-associated liposomes without removing the free drug. DSC measurements and thermograms were recorded at acidic and neutral pH conditions, namely, pH 3.6 and pH 7.0 in order to simulate the internal and external conditions of the liposome.

The main phase transition temperature (Tm), enthalpy (ΔH), heat capacity (Cp1/2), phase transition temperature peak width (Tm_{1/2}), and phase temperature peak temperature (Tp) were measured and the van't Hoff's enthalpy (ΔH_{vH}) and cooperativity (coop) were calculated. These results are detailed in Tables 4 and 5, respectively.

TABLE 4
DSC Parameters of DSPC MLVs with Various Drugs at pH 3.6

| | Tm | Tm | | | | | |
|---|---|---|---|---|---|---|---|
| drug | (° C.) | (K) | ΔHcal | Cpmax | ΔTm_{1/2} | ΔHvH | CU |
| DSPC/ | 54.7 | 327.8 | 9.44 | 8.5 | 0.92 | 769.2 | 81.5 |
| placebo | | | | | | | |
| doxorubicin | 54.5 | 327.7 | 9.28 | 7.4 | 0.94 | 680.5 | 73.3 |
| CKD602 | 54.6 | 327.8 | 11.81 | 7.5 | 1.27 | 542.3 | 45.9 |
| vincristine | 54.6 | 327.8 | 12.77 | 7.1 | 1.42 | 474.9 | 37.2 |
| ciprofloxacin | 54.9 | 328 | 9.98 | 4.5 | 1.4 | 385.6 | 38.6 |
| paclitaxel | 54.5 | 327.6 | 10.6 | 3.5 | 2.27 | 281.7 | 26.6 |

TABLE 5
DSC Parameters of DSPC MLVs with Various Drugs at pH 7.0

| | Tm | | | | | | Tm |
|---|---|---|---|---|---|---|---|
| drug | (C.) | ΔTm_{1/2} | ΔHcal | Cpmax | ΔHv | Coop | (K) |
| DSPC | 54.37 | 0.47 | 9.96 | 14.4 | 1231 | 124 | 327.52 |
| doxorubicin | 54.45 | 0.4 | 9.66 | 16.3 | 1437 | 149 | 327.6 |
| CKD602 | 54.43 | 0.42 | 11.2 | 17.2 | 1308 | 117 | 327.58 |
| vincristine | 53.93 | 1.38 | 11.65 | 6.81 | 496 | 43 | 327.08 |
| paclitaxel | 54.07 | 0.77 | 9.94 | 8.67 | 741 | 75 | 327.22 |

Statistical analysis of the ΔH_{vH} and CU was performed to correlate the data with T_{1/2} utilizing the JMP5.0.1 program. The results are shown in FIGS. 4A-5B. A summary of the results for ΔH_{vH} at pH 3.6 is presented in Tables 6a-6c, below. A summary of the results for ΔH_{vH} at pH 7.0 is presented in Tables 7a-7c, below.

Bivariate scatterplot matrices of correlations of ΔH_{vH} or CU vs. circulation half-life (T_{1/2}) for liposome entrapped drugs at pH 3.6 and 7.0 were prepared 5 and are presented in FIGS. 4A-4B and FIGS. 5A-5B, respectively.

Multivariate scatterplot matrices of correlations of the DSC parameters and the circulation half-life (T_{1/2}) for liposome entrapped drugs at pH 3.6 and 7.0 were prepared and are presented in FIGS. 6A-6B, respectively.

Linear Fit for FIG. 4A
T_{1/2}=−16.43+0.056 ΔH_{vH}

TABLE 6a
Summary of Fit for pH 3.6

| | |
|---|---|
| RSquare | 0.889218 |
| RSquare Adj | 0.852291 |
| Root Mean Square Error | 3.546925 |
| Mean of Response | 15.67 |
| Observations (or Sum | 5 |
| Wgts) | |

TABLE 6b
Analysis of Variance for pH 3.6

| Source | DF | Sum of Squares | Mean Square | F Ratio |
|---|---|---|---|---|
| Model | 1 | 302.94598 | 302.946 | 24.0803 |
| Error | 3 | 37.74202 | 12.581 | Prob > F |
| C. Total | 4 | 340.68800 | | 0.0162 |

TABLE 6c
Parameter Estimates for pH 3.6

| Term | Estimate | Std Error | t Ratio | Prob > \|t\| |
|---|---|---|---|---|
| Intercept | −16.42733 | 6.730503 | −2.44 | 0.0924 |
| Delta | 0.056263 | 0.011465 | 4.91 | 0.0162 |
| HvH | | | | |

TABLE 7a
Summary of Fit for pH 7.0

| | |
|---|---|
| RSquare | 0.920349 |
| RSquare Adj | 0.893799 |
| Root Mean Square Error | 3.007548 |
| Mean of Response | 15.67 |
| Observations (or Sum | 5 |
| Wgts) | |

TABLE 7b
Analysis of Variance for pH 7.0

| Source | DF | Sum of Squares | Mean Square | F Ratio |
|---|---|---|---|---|
| Model | 1 | 313.55196 | 313.552 | 34.6644 |
| Error | 3 | 27.13604 | 9.045 | Prob > F |
| C. Total | 4 | 340.68800 | | 0.0098 |

TABLE 7c
Parameter Estimates for pH 7.0

| Term | Estimate | Std Error | t Ratio | Prob > \|t\| |
|---|---|---|---|---|
| Intercept | −8.054354 | 4.248061 | −1.90 | 0.1542 |
| CU | 0.4289318 | 0.072853 | 5.89 | 0.0098 |

Although the invention has been described with respect to particular embodiments, it will be apparent to those skilled in the art that various changes and modifications can be made without departing from the invention.

1. A method for generating a correlation between at least one thermal property of a liposomal carrier in the presence of a therapeutic agent and a pharmacokinetic property for the liposomal carrier in the presence of the therapeutic agent comprising:
measuring at least one thermal property of said liposomal carrier in the presence of a first therapeutic agent;
measuring at least one thermal property of said liposomal carrier in the presence of a second therapeutic agent;
generating at least one reference correlating a range of values for the pharmacokinetic property with the at least one thermal property.

2. The method of claim 1, wherein said pharmacokinetic property is an in vivo half-life.

3. The method of claim 1, wherein said measuring includes determining the thermal property with an analytical technique

4. The method of claim 3, wherein said analytical technique is a differential scanning calorimeter.

5. The method of claim 3, wherein said thermal property is a phase transition temperature.

6. The method of claim 5, wherein said phase transition (Tₘ) is measured at the peak height.

7. The method of claim 6, wherein the integral under the peak for the phase transition, for the therapeutic agent admixed with the model lipid in about a 1:5 molar ratio, at about pH 3.6, and a DSC scan rate of about 20° C./hour, and (2) calculating the ΔH_{vH} from the equation [(4*R*Tₘ²*Cpₘₐₓ)]/ΔH_{cal}], where R is the universal gas constant (1.9872 cal/mol*K) and the enthalpy corresponds to the integral.

8. A method for predicting a pharmacokinetic property of a liposomal carrier in the presence of a therapeutic agent, comprising:
selecting the liposomal carrier;
determining at least one thermal property of the liposomal carrier in the presence of the therapeutic agent by an analytical technique;
comparing said at least one thermal property to a generated correlation for said liposomal carrier; and
determining the pharmacokinetic property of the liposomal carrier in the presence of the therapeutic agent.

9. The method of claim 8, wherein said pharmacokinetic property is an in vivo blood circulation half-life.

10. The method of claim 8, wherein said analytical technique is differential scanning calorimetry.

11. The method of claim 8, wherein said at least one thermal property is a calculated van't Hoff enthalpy value (ΔH_{vH}).

12. The method of claim 8, wherein said ΔH_{vH} is calculated from the equation [(4*R*Tₘ²*Cpₘₐₓ)]/ΔH_{cal}], where R is the universal gas constant (1.9872 cal/mol*K), Tₘ is the phase transition of the therapeutic agent in the presence of the lipid, Cpₘₐₓ is the heat capacity at the peak of the transition, and the calorimetric enthalpy, ΔH_{cal} is the integral under the peak for the phase transition with Tₘ, Cpₘₐₓ, and ΔH_{cal} being determined from a differential scanning calorimetry trace for a mixture of the therapeutic agent and the liposomal carrier, at pH 3.6 and at a scan rate of 20° C./hour.
